(19) United States
(12) Patent Application Publication
(10) Pub. No.: US 2011/0283819 A1
(43) Pub. Date: Nov. 24, 2011

(54) Repetitive Motion Skill Building Device

(76) Inventor: Rick Paulus, Cedar Grove, WI (US)

(21) Appl. No.: 13/111,846

(22) Filed: May 19, 2011

Related U.S. Application Data
(60) Provisional application No. 61/346,647, filed on May 20, 2010.

(51) Int. Cl. A61B 5/11; B23P 11/00

(52) U.S. Cl. 73/865.4; 29/428

(57) ABSTRACT

The present invention is a repetitive motion skill building device with a weighted base and a movable arm which simulates the chest or arm of another player. The movable arm is capable of swinging in all directions and will return to its original location. The height of the movable arm is adjustable allowing it to be used for players of varying ages, heights and skill levels. The repetitive motion skill building device may be used for arm and hand drills.

### CROSS-REFERENCE TO RELATED APPLICATIONS

This application claims priority to U.S. Provisional Application No. 61/346,647 filed on May 20, 2010.

### FIELD OF INVENTION

The present invention relates to the field of football training devices and more particularly to a repetitive motion skill building device.

### BRIEF DESCRIPTION OF THE DRAWINGS

FIG. 1 illustrates an exploded view of an exemplary embodiment of a repetitive motion skill building device.

FIGS. 2 and 3 illustrate perspective views of an exemplary embodiment of a repetitive motion skill building device.

FIG. 4 illustrates a rear view of an exemplary embodiment of a repetitive motion skill building device.

FIG. 5 illustrates a side view of an exemplary embodiment of a repetitive motion skill building device.

FIG. 6 illustrates a front view of an exemplary embodiment of a repetitive motion skill building device.

FIG. 7 illustrates a cross-sectional view of an exemplary embodiment of a repetitive motion skill building device taken along line VII of FIG. 6.

FIG. 8 illustrates an exemplary embodiment of padding for a repetitive motion skill building device.

### BACKGROUND

It is estimated that there are more than one million interscholastic football participants in the United States. Football skill building devices that simulate other players for teaching and training participants are known in the art. For example, blocking sleds are often used to develop strength and blocking techniques and are geared toward developing offensive and defensive players. Blocking sleds, however, are heavy, bulky pieces equipment that typically cost thousands of dollars depending on the size and the number of men on the sled. Blocking sleds are available in two different sizes, youth level and secondary/post-secondary; however, the frames are not adjustable to accommodate players of varying heights/ages requiring the purchase of multiple blocking sleds.

### SUMMARY OF THE INVENTION

The present invention is a repetitive motion skill building device comprised of a weighted base, outer and inner support components, a spring assembly, and a movable arm which simulates the chest or arm of another player. The movable arm is capable of swinging in all directions and will return to its original location. The height of the movable arm is adjustable by raising the inner support component to which the movable arm is secured. Having a movable arm height that is adjustable allows a single repetitive motion skill building device to be used for players of varying ages, heights, and skill levels. The repetitive motion skill building device may be used for arm and hand drills (e.g., to teach rip and swim moves) as well as for fumbling drills.

It is desirable to have a repetitive motion skill building device which is adjustable, allowing a single device to be used by players of varying ages, heights, and skill levels.

It is further desirable to have a repetitive motion skill building device that is lightweight, easy to transport on a practice field, and capable of being dissembled for storage.

### DETAILED DESCRIPTION OF INVENTION

For the purpose of promoting an understanding of the present invention, references are made in the text to exemplary embodiments of a repetitive motion skill building device, only some of which are described herein. It should be understood that no limitations on the scope of the invention are intended by describing these exemplary embodiments. One of ordinary skill in the art will readily appreciate that alternate but functionally equivalent materials, components, and placement may be used. The inclusion of additional elements may be deemed readily apparent and obvious to one of ordinary skill in the art. Specific elements disclosed herein are not to be interpreted as limiting, but rather as a basis for the claims and as a representative basis for teaching one of ordinary skill in the art to employ the present invention.

It should be understood that the drawings are not necessarily to scale; instead, emphasis has been placed upon illustrating the principles of the invention. In addition, in the embodiments depicted herein, like reference numerals in the various drawings refer to identical or near identical structural elements.

Moreover, the terms “substantially” or “approximately” as used herein may be applied to modify any quantitative representation that could permissibly vary without resulting in a change in the basic function to which it is related.

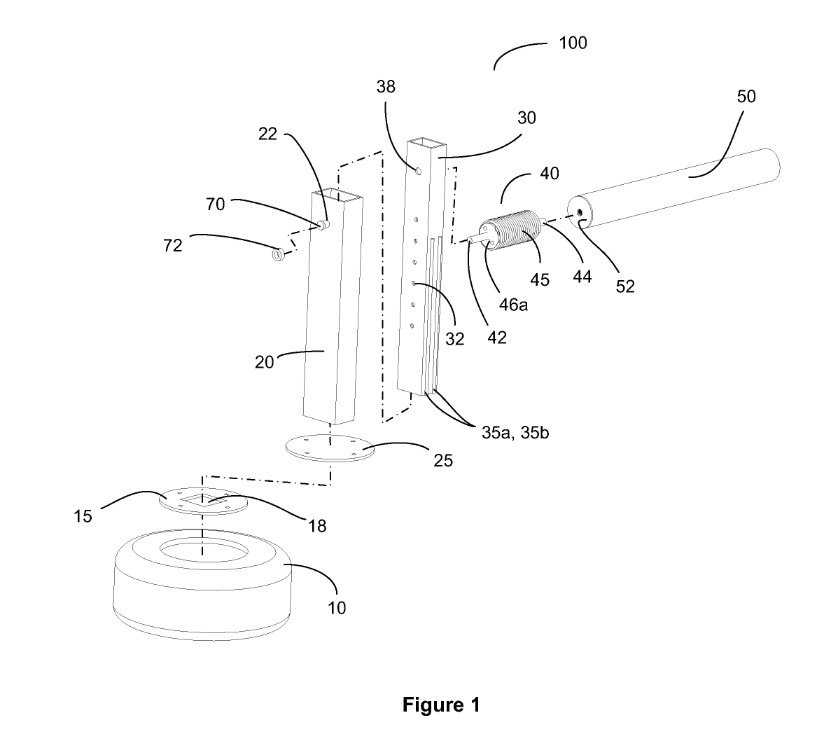

FIG. 1 illustrates an exploded view of an exemplary embodiment of repetitive motion skill building device 100. Repetitive motion skill building device 100 is comprised of base 10, base plate 15, outer support component 20, outer support component plate 25, inner support component 30, spring assembly 40, and movable arm 50.

In the embodiment shown, base 10 is hollow and base plate 15 is molded into and secured to the top of base 10. Base plate 15 has center aperture 18 which allows sand or another suitable material (e.g., gravel) to be added to the inside of base 10 to weight it for use.

In the embodiment shown, base 10 is circular with radial edges and has a height of approximately 6.5 inches and a diameter of approximately 16 inches. The radial edges help to prevent injury if a player were to fall onto base 10 or trip on an edge. In various other embodiments, base 10 may be square, rectangular, or any other shape and/or may have square edges.

In the embodiment shown, base 10 is comprised of a plastic material, including, but not limited to polypropylene, acrylonitrile butadiene styrene (ABS), and polyethylene. In various embodiments, base 10 may be comprised of another type of material, such as rubber. In still other embodiments, base 10 may not be hollow, but rather is solid and/or weighted, eliminating the need for the user to fill the base with sand or another material to weigh down base 10.

In the embodiment shown, base plate 15 is comprised of steel and is secured to base 10 by an in-molded manufacturing process, which eases assembly and imparts strength. In various other embodiments, base plate 15 may be comprised of another material, such as plastic, and/or may be secured to base 10 using another means known in the art, such as fasteners. In still other embodiments, base 10 and base plate 15 may be manufactured as a single unit.

In the embodiment shown, outer support component 20 has a rectangular cross section and includes spring pin aperture 22 for retractable spring pin 70. Retractable spring pin spacer 72 is welded to the outer surface of outer support component 20 around spring pin aperture 22. Outer support component 20 is secured to the top of outer support component plate 25. Outer support component plate 25 is then secured to base plate 15 by inserting screws or another type of fastener through corresponding apertures in outer support component plate 25 and base plate 15, securing outer support component 20 to base 10.

In the embodiment shown, spring pin aperture 22 is centered on the face of outer support component 20 and is approximately 2.5 inches from the top of outer support component 20 and approximately 18.5 inches from the bottom of outer support component 20. In various other embodiments, the location of spring pin aperture 22 may vary. In various other embodiments, a clevis pin, split pin, or similar component may be used in place of retractable spring pin 70 and/or retractable spring pin spacer 72 may be omitted.

Inner support component 30 is inserted into outer support component 20. In the embodiment shown, inner support component 30 has a rectangular cross section and dimensions which are slightly smaller than those of outer support component 20, allowing inner support component 30 to slide vertically within outer support component 20.

In the embodiment shown, outer support component 20 is approximately 4 inches by 21 inches with a thickness of approximately 0.188 inches and inner support component 30 is approximately 3 inches by 23.31 inches with a thickness of approximately 0.188 inches. In various other embodiments, the dimensions and/or thickness of outer support component 20 and inner support component 30 may vary.

Inner support component 30 includes a plurality of securing apertures 32 which allow for incremental height adjustment of inner support component 30 and movable arm 50, which is secured to inner support component 30. To lock inner support component 30 in position, retractable spring pin 70 is threaded into retractable spring pin spacer 72, which is welded to the outer surface of outer support component 20. The nose of retractable spring pin 70 is inserted into spring pin aperture 22 in outer support component 20, and will then locate and secure in one of securing apertures 32 in inner support component 30.

To adjust the height of movable arm 50, retractable spring pin 70 is retracted from its current securing aperture 32. Inner support component 30 and movable arm 50 are then vertically raised or lowered to the desired height and retractable spring pin 70 is reinserted into a securing aperture 32, locking inner support component 30 and movable arm 50 into place.

Inner support component 30 further includes spacers 35a, 35b, 35c, and 35d (spacers 35c and 35d are located on the side of inner support component 30 opposite spacers 35a and 35b and are not visible). Spacers 35a, 35b, 35c, and 35d allow for play in the X direction between inner support component 30 and outer support component 20, ensuring unrestricted linear movement of inner support component 30. Spacers 35a, 35b, 35c, and 35d also allow any dirt that gets between inner support component 30 and outer support component 20 to drop between them, preventing the dirt from restricting sliding of inner support component 30.

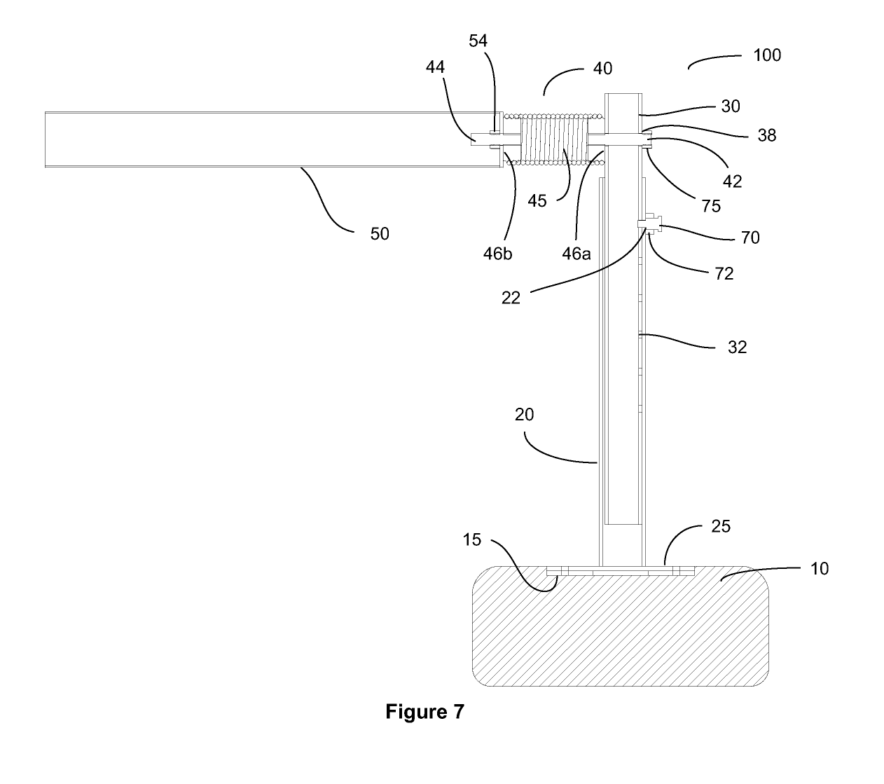

In the embodiment shown, spring assembly 40 is comprised of spring 45, threaded rod 42, threaded rod 44, and threaded plugs 46a, 46b (threaded plug 46b not visible). Threaded plugs 46a, 46b are turned into the ends of spring 45 and locked. Threaded rod 42 is then inserted into threaded plug 46a and threaded rod 44 into threaded plug 46b. Arm cap 52 and nut 54 (not visible, secured to the inner surface of arm cap 52, see FIG. 7) are welded to one end of movable arm 50 and then movable arm 50 is threaded onto threaded rod 44, securing movable arm 50 to spring assembly 40.

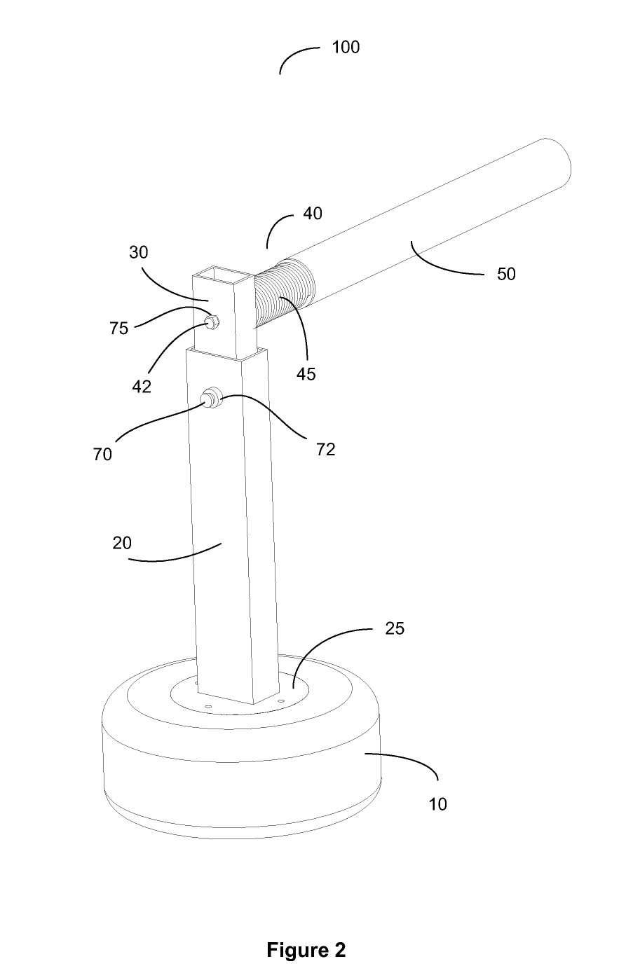

Threaded rod 42 is assembled through spring assembly aperture 38 in inner support component 30. Nyloc nut 75 (not shown, see FIG. 2) or similar locknut component is then threaded onto the end of threaded rod 42 which protrudes from outer support component 20, securing spring assembly 40 and movable arm 50 to inner support component 30.

In the embodiment shown, all of the components, with the exception of base 10, are comprised of steel and are e-coated for rust protection. In various other embodiments, one or more components may be comprised of another material or combination of materials. For example, outer support component 20 and inner support component 30 may be comprised of extruded plastic.

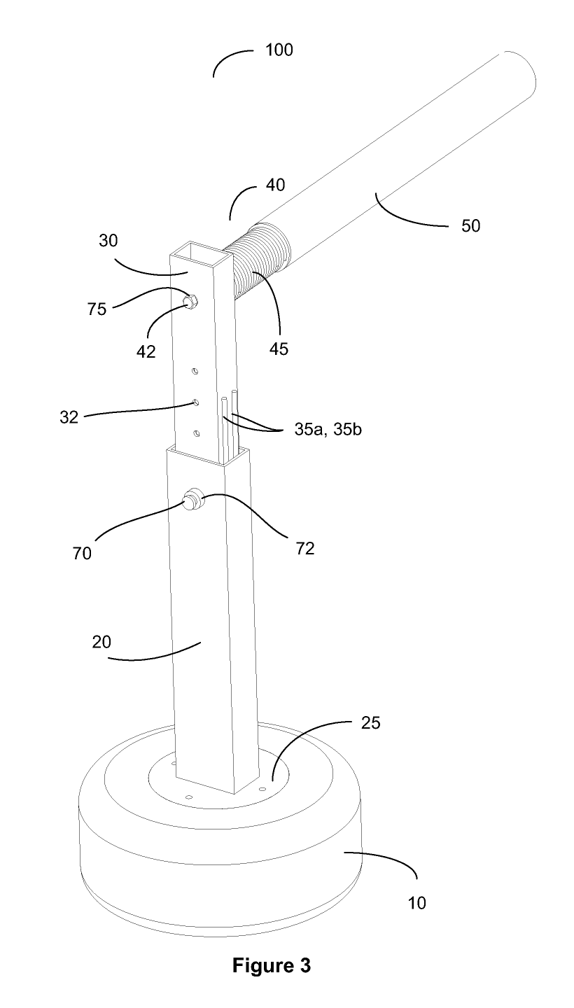

FIGS. 2 and 3 illustrate perspective views of an exemplary embodiment of repetitive motion skill building device 100 comprised of base 10, base plate 15, outer support component 20, outer support component plate 25, inner support component 30, spring assembly 40, and movable arm 50.

Repetitive motion skill building device 100 is stabilized by adding weight to base 10 (e.g., filling base 10 with sand) before securing outer support component plate 25 to base plate 15 (not visible). A weighted base 10 will ensure that outer support component 20 and inner support component 30 remain in the upright position, when movable arm 50 is in motion.

The height of movable arm 50 may be adjusted to allow a single repetitive motion skill building device 100 to accommodate players of varying ages and heights. In FIG. 2, movable arm 50 is in the fully lowered position, and in FIG. 3, movable arm 50 is in a partially raised position.

In the embodiment shown, movable arm 50 is locked in a horizontal position. To adjust the height of movable arm 50, retractable spring pin 70 is retracted, releasing it from its current securing apertures 32. Movable arm 50 and inner support component 30 may be then raised or lowered. When the desired height of movable arm is reached, retractable spring pin 70 is reinserted into another securing aperture 32, securing movable arm 50 and inner support component 30 to outer support component 20.

In the embodiment shown, inner support component 30 has six securing apertures 32, the first of which is located approximately 7.06 inches from the top of inner support component 30 and approximately 16.25 inches from the bottom of inner support component 30. Securing apertures 32 are positioned approximately 2.0 inches apart, allowing for incremental height adjustment to accommodate players of varying heights. In various other embodiments, inner support component 30 may contain more or fewer securing apertures 32 located closer or further from the top/bottom of inner support component 30, and/or which may be located closer or further apart.

In the embodiment shown, inner support component 30 and movable arm 50 may be completely removed from outer support component 20 and base 10 for storage.

In the embodiment shown, spring assembly 40 allows movable arm 50 to swing in all directions and return to its original resting position. In an exemplary embodiment, movable arm 50 will return to its original resting position without springing back with a large of amount of force, which may be dangerous to the player. In the embodiment shown, spring 45 is a helical spring which has a rate of approximately 20 to 23 pounds per inch; however, in various other embodiments, spring 45 may have a rate ranging from 17 to 35 pounds per inch.

Movable arm 50 of repetitive motion skill building device 100 simulates the chest or arm of another stationary player or one coming toward the player and allows the player to practice repetitive motions used in a football game (e.g., rip move, swim move). Repetitive motion skill building device 100 teaches players how to use their arms and hands to defend against other players. When used to practice repetitive motions, the height of movable arm 50 is adjusted so that it is positioned at the center line of the player's shoulder or slightly above when he or she is in a three-point stance.

Repetitive motion skill building device 100 may also be used in fumbling drills. For example, a series of repetitive motion skill building devices 100, may be placed in a zigzag or offset pattern so that the player can run through the repetitive motion skill building devices 100. The heights of repetitive motion skill building devices 100 may be varied to simulate players of varying heights.

In the embodiment shown, movable arm 50 has a diameter of approximately 3 inches and a length of approximately 24.5 inches. In various embodiments, the length of movable arm 50 may be adjusted to increase/decrease the amount of tension and the strength required to move movable arm 50. The longer the arm, the easier the arm will be able to move. For example, a longer movable arm 50 may be used for younger children while a shorter arm may be used for high school or college level players.

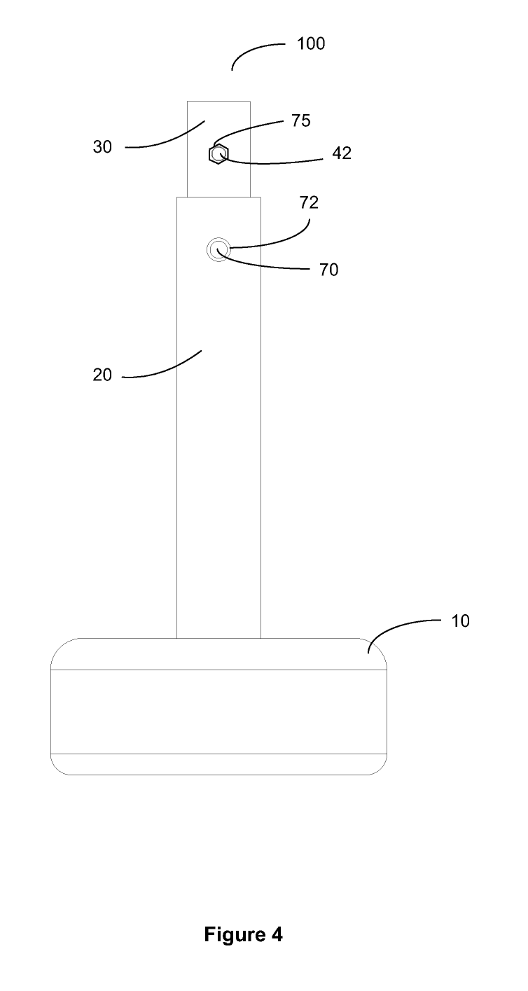

FIG. 4 illustrates a rear view of an exemplary embodiment of repetitive motion skill building device 100. Visible in the embodiment shown are base 10, outer support component 20, inner support component 30, retractable spring pin 70, retractable spring pin spacer 72, the end of threaded rod 42, and nyloc nut 75.

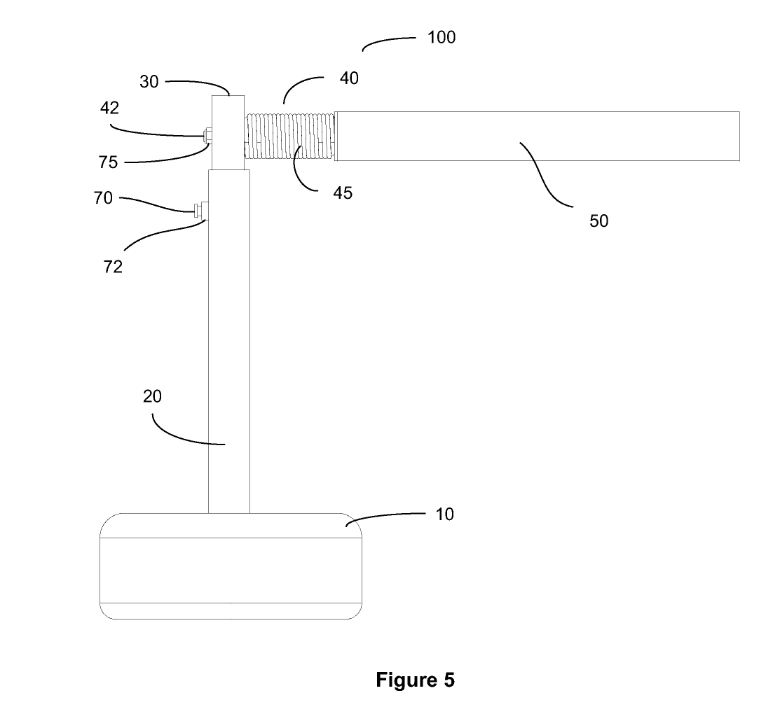

FIG. 5 illustrates a side view of an exemplary embodiment of repetitive motion skill building device 100. Visible in the embodiment shown are base 10, outer support component 20, inner support component 30, spring assembly 40, spring 45, movable arm 50, retractable spring pin 70, retractable spring pin spacer 72, the end of threaded rod 42, and nyloc nut 75.

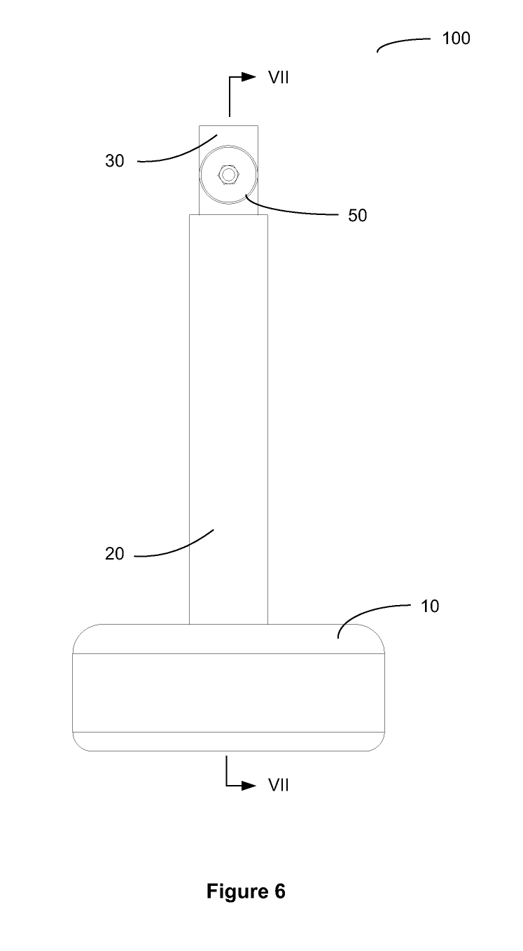

FIG. 6 illustrates a front view of an exemplary embodiment of repetitive motion skill building device 100. Visible in the embodiment shown are base 10, outer support component 20, inner support component 30, and movable arm 50.

FIG. 7 illustrates a cross-sectional view of an exemplary embodiment of repetitive motion skill building device 100 taken along line VII of FIG. 6. Visible in the embodiment shown are base 10, base plate 15, outer support component 20, outer support component plate 25, inner support component 30, spring assembly 40, spring 45, threaded plugs 46a, 46b, threaded rods 42, 44, nyloc nut 75, nut 54, spring assembly aperture 38, movable arm 50, retractable spring pin 70, retractable spring pin spacer 72, and securing apertures 32.

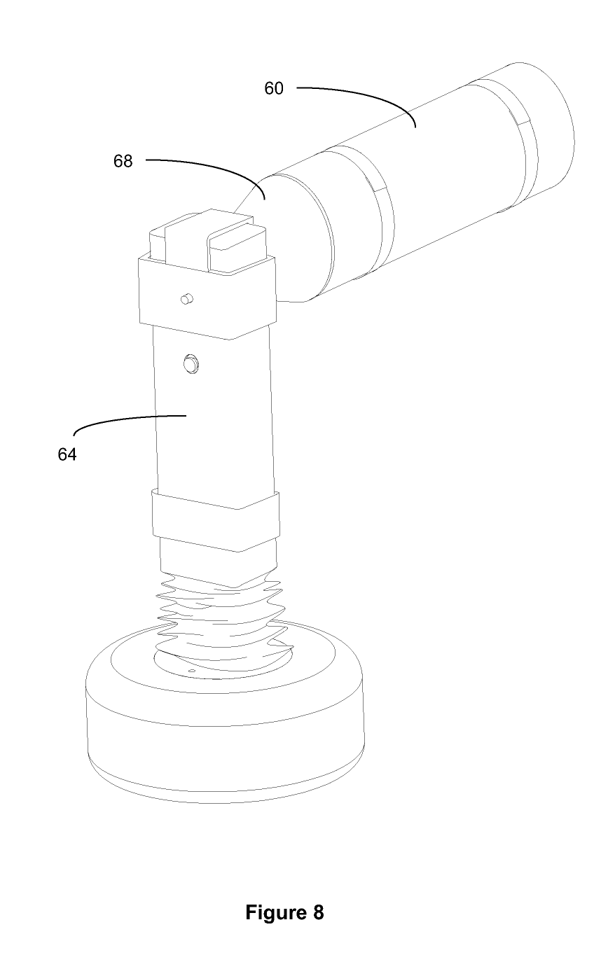

FIG. 8 illustrates an exemplary embodiment of padding 60, 64, and 68 for repetitive motion skill building device 100. In the embodiment shown, movable arm 50 is surrounded by padding 60, outer support component 20 and inner support component 30 are surrounded padding 64, and spring assembly 40 is surrounded by padding 68.

In the embodiment shown, padding 60, 64, and 68 is comprised of foam covered in plastic or vinyl and is secured around movable arm 50, outer and inner support components 20, 30, and spring assembly 40, respectively, using hook-and-loop fasteners. Padding 64 further includes an opening which allows access to retractable spring pin 70 and accordion folds at the bottom to accommodate height adjustment of inner support component 30 and movable arm 50. In various other embodiments, padding 60, 64, and 68 is comprised of another material and/or is secured using hooks, buckles, threading, or another type of fastener. In still other embodiments, padding 60, 64, and 68 may be customized (e.g., with school colors).

What is claimed is:

1. A repetitive motion skill building apparatus comprised of:
a weighted base;
a hollow outer support component having a rectangular cross section, a first end of said outer support component is secured to said weighted base, said outer support component having a retractable spring pin aperture positioned near a second end;
a hollow inner support component having a rectangular cross section which moves slidably inside said outer support component, said inner support component having a spring assembly aperture and a plurality of securing apertures which allow for incremental height adjustment of a movable arm;
a retractable spring pin, said retractable spring is inserted through said spring pin aperture in said outer support component and into one of said plurality of securing apertures in said inner support component to secure said inner support component to said outer support component;
a spring assembly having a first end and a second end, said first end of said spring assembly is inserted into said spring assembly aperture in said inner support component; and
a movable arm, said movable arm secured to said second end of said spring assembly.

2. The apparatus of claim 1 which further includes a first plate secured to said weighted base and a second plate secured to the bottom of said outer support component, said second plate is secured to said first plate using a plurality of fasteners.

3. The apparatus of claim 1 wherein said weighted base is weighted with sand.

4. The apparatus of claim 1 wherein said weighted base is circular with radial edges.

5. The apparatus of claim 1 which further includes a retractable spring pin spacer welded to said outer support component around said retractable spring pin aperture.

6. The apparatus of claim 5 wherein said retractable spring pin is threaded into said retractable spring pin spacer.

7. The apparatus of claim 1 wherein said inner support component further includes a plurality of spacers to ensure unrestricted linear movement of said inner support component.

8. The apparatus of claim 1 wherein said spring assembly is comprised of:
a spring;
a first threaded plug turned into a first end of said spring;
a second threaded plug turned into a second end of said spring;
a first threaded rod threaded into said first threaded plug; and
a second threaded rod threaded into said second threaded plug.

9. The apparatus of claim 8 wherein a nyloc nut is threaded onto the end of said first threaded rod after said first threaded rod is threaded through said spring assembly aperture in said inner support component.

10. The apparatus of claim 8 wherein said spring has a rate ranging from 20 to 23 pounds per inch.

11. The apparatus of claim 1 wherein said weighted base is comprised of plastic.

12. The apparatus of claim 1 wherein said outer support component, said inner support component, said spring assembly, said retractable spring pin, and said movable arm are comprised of steel coated for rust protection.

13. The apparatus of claim 1 wherein said inner support component has six securing apertures.

14. The apparatus of claim 13 wherein said six securing apertures are positioned two inches apart to allow for incremental height adjustment of said movable arm.

15. The apparatus of claim 1 wherein said movable arm is capable of moving in all directions.

16. The apparatus of claim 1 wherein said movable arm has a diameter of 3 inches and a length of 24.5 inches.

17. The apparatus of claim 1 wherein said outer support component, said inner support component, said spring assembly, and said movable arm are surrounded by padding.

18. A method of assembling a repetitive motion skill building apparatus comprising the steps of:
inserting sand into a base through an aperture in a first plate secured to the top of said base to add weight to said base;
securing an outer support component to a second plate;
securing said second plate to said first plate;
inserting an inner support component into said outer support component;
securing said inner support component to said outer support component by inserting a retractable spring pin through an aperture in said outer support component into one of a plurality of securing apertures in said inner support component;
assembling a spring assembly by turning a first threaded plug into a first end of a spring, turning a second threaded plug into a second end of said spring, threading a first rod into said first threaded plug, and threading a second rod into said second threaded plug;
securing a movable arm to said second rod; and
inserting said first rod into an aperture in said inner support component.

19. The method of claim 18 which further includes threading a nyloc nut on the end of said first rod.

20. The method of claim 18 which further includes the step of adding padding around said outer support component and said inner support component, said spring assembly, and said movable arm.
